(19) United States
(12) Patent Application Publication
(10) Pub. No.: US 2005/0182364 A1
(43) Pub. Date: Aug. 18, 2005

(54) Ice pain management device and method

(76) Inventor: Corey A. Burchman, York, PA (US)

Correspondence Address: Jeffrey S. Habib, Esq.;Hooker & Habib, P.C., Suite 304, 100 Chestnut Street, Harrisburg, PA, 17101, US

(21) Appl. No.: 10/777,736

(22) Filed: Feb. 12, 2004

(51) Int. Cl. A61M 5/00

(52) U.S. Cl. 604/112; 606/20; 604/500

(57) ABSTRACT

An inexpensive and disposable cooling device for relieving pain associated with hypodermic injection that can be used with any hypodermic syringe includes a body and a sterile cooling medium within the body. A removable cover closes the body and maintains the sterility of the cooling medium before use. The device can be used to simultaneously cool a number of injection sites, eliminating the additional time required to serially cool the multiple injection sites.

### FIELD OF THE INVENTION

The invention relates to devices that relieve pain associated with hypodermic injection and methods for relieving pain associated with hypodermic injection.

### DESCRIPTION OF THE PRIOR ART

The pain associated with hypodermic needle injection is often a reason given for delaying or avoiding health care.

It is known, however, that cooling the skin de-sensitizes the skin and enables hypodermic injection with little or no pain.

Cooling devices have been developed that are used in combination with hypodermic syringes to cool the skin immediately prior to injection. Such devices tightly hold the syringe and include a syringe support that is pressed against the skin. The syringe support is pre-chilled to well-below room temperature and cools the skin upon contact. After sufficient time has passed to de-sensitize the skin (typically about 20 seconds), the injection is made.

Although useful, conventional cooling devices have a number of disadvantages. The syringe support is adapted to receive hypodermic syringes of a specific size, and is made of metal for efficient heat transfer between the skin and the support. This makes conventional cooling devices relatively expensive to manufacture, increasing purchase cost. A number of different supports must be kept on hand for use with different-sized syringes, increasing inventory cost. The supports are not disposable and are re-used, increasing storage costs. And the supports must be sterilized by medical staff before use, increasing operating cost.

Furthermore, conventional cooling devices are capable of only cooling the skin around a single injection site. Many procedures, however, require multiple injections. A separate cooling device for each injection must be provided, further increasing inventory cost. This also presents a problem not previously recognized, that serial cooling of multiple sites increases patient anxiety.

To illustrate, a typical series of lumbar spine injections may require eight injections spread over an area measuring 20 cm by 15 cm. Each injection requires the use of a separate cooling device to cool an injection site, so eight devices must be provided. The injections must be spaced apart a minimum of 20 seconds to serially apply the cooling devices against the skin, adding at least 160 seconds to the procedure and forcing an already anxious patient to endure a substantially longer procedure.

Therefore there is a need for an improved cooling device to reduce the pain associated with hypodermic injection, and an improved method for cooling multiple injection sites. The device should be inexpensive to manufacture and be disposable, be usable with any hypodermic syringe, and not require sterilization prior to use. The method should enable cooling the skin around multiple injection sites without materially increasing the time to carry out the injections.

### SUMMARY OF THE INVENTION

The present invention is an improved cooling device to reduce the pain associated with hypodermic injections. The device is inexpensive to manufacture and can be used with any hypodermic syringe. The device is also disposable and does not have to be sterilized by medical staff before use.

Embodiments of the device can be used to simultaneously cool an additional number of injection sites. The additional time required to serially cool multiple injection sites is thereby eliminated.

A cooling device in accordance with the present invention includes a body having an open end and an interior wall extending inwardly from the open end. The interior wall partially defines a reservoir that extends into the body. A cover removably seals and closes the open end of the body, the cover and interior wall enclosing the reservoir. A cooling medium is in the reservoir, the cooling medium being retained in the body when the cover is removed.

The body, cover and cooling medium are capable of being cooled to an application temperature substantially below room temperature. Removing the cover exposes the cooled cooling medium such that placing the open end of the body against the skin causes sufficient heat transfer between the cooling medium and the skin to materially de-sensitize the skin.

In preferred embodiments the body is molded as a single piece of plastic. This enables the device to be inexpensive and disposable. The cooling medium in possible embodiments is sterile water that becomes ice at the application temperature (0 degrees Centigrade or below). Sterile water or ice is inexpensive and readily available. Other cooling mediums or application temperatures can be used.

Alternative embodiments of the device can be sized to cover and simultaneously cool a number of injection sites. The device can be configured to cool multiple sites extending substantially along a straight line or distributed within an area. The cooling medium is applied against an area of skin having a perimeter that encloses the injection sites. The cooling medium is capable of cooling the skin sufficiently to simultaneously de-sensitize each of the injection sites.

The cooling medium is maintained against the area of skin for a length of time sufficient to materially de-sensitize the entire area of skin. The hypodermic injections are serially administered at the injection sites while the area of skin remains de-sensitized without additional application of a cooling medium between injections. Even if only some of the injection sites were simultaneously cooled, a substantial savings of time and patient pain and anxiety would be achieved.

Other objects and features of the invention will become apparent as the description proceeds, especially when taken in conjunction with the accompanying 2 drawing sheets illustrating six embodiments of the invention.

### BRIEF DESCRIPTION OF THE DRAWINGS

FIG. 1 is an elevation view of a first embodiment cooling device in accordance with the present invention;

FIG. 2 is a sectional view of the cooling device shown in FIG. 1 taken along line 2—2 of FIG. 1;

FIG. 3 is an enlarged view of region A shown in FIG. 2;

FIG. 4 is a representational view of an injection site cooled by the cooling device shown in FIG. 1;

FIG. 5 is an elevation view of a second embodiment cooling device in accordance with the present invention;

FIG. 6 is a representational view of multiple injection sites cooled by the cooling device shown in FIG. 5;

FIG. 7 is an end view of the applicator end of a third embodiment cooling device in accordance with the present invention;

FIG. 8 is an end view of the applicator end of a fourth embodiment cooling device in accordance with the present invention;

FIG. 9 is a sectional view similar to FIG. 2 of a fifth embodiment cooling device in accordance with the present invention; and

FIG. 10 is a view similar to FIG. 3 of a sixth embodiment cooling device in accordance with the present invention.

### DESCRIPTION OF THE PREFERRED EMBODIMENTS

FIGS. 1-3 illustrate a first embodiment cooling device 10 in accordance with the present invention. The device 10 is intended for cooling an area of skin around a single injection site.

Device 10 includes a tubular body 12 holding cooling medium 14 within the body. A removable cover 16 sealingly closes the body 12 and prevents the release of cooling medium from the device prior to use.

The body 12 is preferably molded or otherwise formed as a single piece of plastic or other insulating material, and extends between an open end 18 and a closed end 20. An applicator portion 22 is formed on the open end of the body, with handle portion 24 extending from the applicator to the closed end. Inner body wall 26 extends inwardly from the open end of the body through the applicator portion 22 and partially defines a reservoir 28 that holds the cooling medium 14. In the illustrated embodiment the reservoir also extends through the handle portion 24. A circumferential lip 30 immediately adjacent the open end of the body extends into the reservoir 28 from the inner wall and retains the cooling medium 14 in the body after the cover 16 is removed.

Cover 16 is formed from substantially planar sheet material, such as foil, that is attached to the open end of the body 12 by an adhesive joint 32. The joint 32 seals the entire circumference between the body and the cover. The cover 16 and body interior wall 26 enclose the reservoir 28.

Cooling medium 14 is sterile water, and the body 12 is sterilized prior to being filled with the cooling medium 14. The side of the cover 16 facing the cooling medium 14 is also sterilized so that the cover 16 protects the cooling medium from the ambient environment and maintains a sterile environment within the reservoir 28.

Prior to use the device 10 is placed in a freezer, standing on the cover 16. The water freezes to ice, the ice formed with a flat lower surface against the cover 16. The volume of the reservoir 28 is sufficient to accommodate expansion of the ice without deforming the cover 16 or the body 12.

It is anticipated that the device 10 will be provided in an entirely sterile condition as described, and come prepackaged as a ready-to-use item in a sterile package or “peel pack” containing a number of the devices 10. The packaging would facilitate the placement of a device 10, when frozen, directly onto a sterile surgical field.

In use, the device 10 is removed from the freezer and grasped by the handle. The handle extends away from the ice 14 and enables grasping the device 10 without substantial heat transfer through the handle. Cover 16 is peeled away from the body 12, exposing the ice 14. The exposed ice 14 is sterile, having been protected by the cover, and no sterilization by medical staff is required.

Lip 30 functions to retain the ice in the body after the cover 16 is removed and prevents the ice from falling out of the body. If the ice sufficiently adheres to the body wall 26, the adhesion can function to retain the ice in the body and the lip 30 can be omitted.

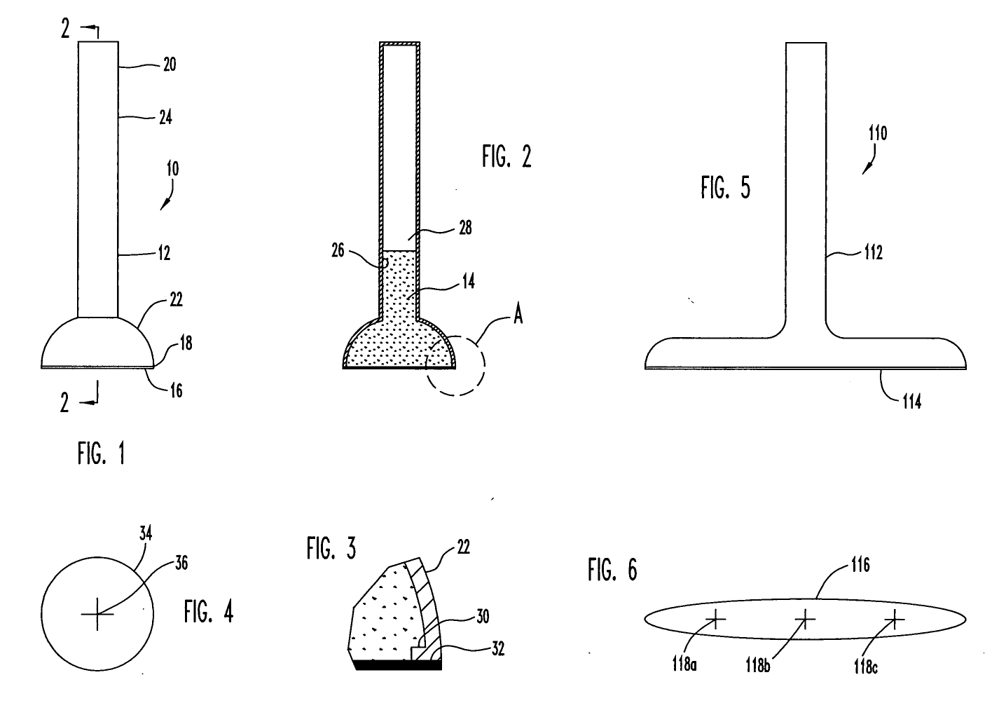

The applicator portion 22 is pressed against the skin at the injection site so that the sterile ice 14 cools the skin. As shown in FIG. 4, the free end of the applicator portion 22 is shaped to chill or cool a circular area 36 centered on an injection site 38. The ice has the thermal capacity to de-sensitize the area 36, and is typically pressed against the skin for about 20 seconds to achieve the desired therapeutic effect.

The device 10 is then removed and a hypodermic injection is made in the conventional manner. The device 10 is disposed of after use.

The device 10 is configured for cooling a single injection site. A number of devices 10 can be serially used to cool a number of spaced-apart injection sites.

FIG. 5 illustrates a second embodiment cooling device 110 formed in accordance with the present invention. The device 110 is intended to simultaneously cool a number of injection sites. Device 110 is similar to device 10, having a body 112 and a removable cover 114 enclosing cooling medium (not shown). The applicator end of the body is shaped for chilling an elongated area of skin 116 containing a number of injection sites 118a, 118b and 118c (see FIG. 6). After the entire area 116 is sufficiently cooled, serial hypodermic injections are administered at the now de-sensitized injection sites. The skin remains de-sensitized without additional cooling between injections.

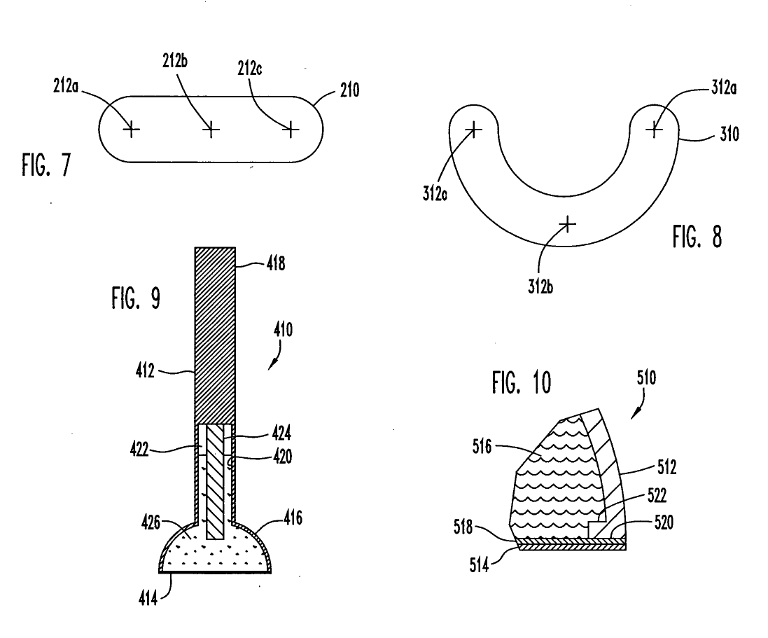

FIGS. 7 and 8 illustrate additional applicator shapes that can be used in alternative embodiments of the present invention. The injector sites shown in the figures are intended to be representative of multiple injection sites and not limiting to the scope of the invention.

FIG. 7 illustrates an oval-shaped applicator end 210 that can be sized to cool a single injection site or to simultaneously cool two or more injection sites such as sites 212a, 212b, and 212c. The oval shape is especially useful for single injection sites if infiltration of the medicant sub- and intradermally occurs that would otherwise also cause pain. For example, infiltration of local anesthetic used for carpal tunnel release typically measures about 10 cm by 5 cm. The larger applicator area cools and de-sensitizes infiltrated skin away from the injection site that would otherwise be unaffected using the applicator 10.

FIG. 8 illustrates a “C” shaped applicator end 310 (“C” shaped is intended to include “U” shapes, crescent shapes, horseshoe shapes, and other curved shapes). This shape is useful for areas, such as the breast, where injection sites extend along semi-lunar, semi-circular, or elliptical paths. For example, shape 310 may be designed to partially surround a nipple that is the typical 5 cm to 6 cm in diameter and cover injection sites 312a, 312b, and 312c.

FIG. 9 illustrates a fifth embodiment cooling device 410 similar to the device 10 that omits the lip 30 and has an alternative means for retaining the ice when the cover is removed. The device 410 has a body 412 and a removable cover 414 like the cover 16. The body 412 includes an applicator portion 416 similar to the applicator portion 22, and a handle portion 418 that define the inner body wall 420 partially enclosing reservoir 422. A retaining rod 424 extends from the wall 420 at the top of the reservoir and extends into cooling medium 426. The water 426 freezes on the retaining rod 420 so that when the cover is removed the ice remains attached to the rod 420. In other embodiments the lip and retaining rod can be used together.

FIG. 10 illustrates a portion of a sixth embodiment cooling device 510 having a body 512 and removable outer cover 514 sealing the open end of the body and maintaining the sterility of a cooling medium 516. A sterile inner cover 518 extends across the open end of the body and is permanently attached to the body by adhesive joint 520. The outer surface of lip 522 (formed like lip 30), forms part of the joint 520 and provides additional adhesive area between the body and the inner cover.

The device 510 is cooled below room temperature to the desired application temperature. The outer cover 516 is removed immediately prior to use and exposes the sterile inner cover 518. In this embodiment the adhesive joint 520 functions to retain the cooling medium 516 in the body when the outer cover 514 is removed.

The device 510 is pressed against the skin with the inner cover 518 engaging the skin. Heat transfer occurs through the inner cover 518, and so the inner cover 518 itself forms part of the cooling medium. The inner cover 518 is sufficiently thin to not substantially impede heat transfer with the skin, or can be made from a heat-conductive material.

The cooling medium 516 may include a water/glycol mixture or a water/alcohol mixture that remains liquid at application temperature. In other embodiments ice or some other freezable substance that is solid at application temperature can be in the device 10, with the inner cover containing any melt liquid that might be generated.

The illustrated embodiments use a cover formed from sheet material that adheres to the end of the device body or to an inner cover. In other possible embodiments the cover can be attached in other ways known in the art, including frictional engagement or with the use of threaded connections.

Other cooling mediums that can be used with the present invention include gels, such as a single phase change material (PCM), used to regulate the temperature of products.

While I have illustrated and described preferred embodiments of my invention, it is understood that this is capable of modification, and I therefore do not wish to be limited to the precise details set forth, but desire to avail myself of such changes and alterations as fall within the purview of the following claims.

1. A device for cooling and thereby de-sensitizing an area of human skin immediately prior to hypodermic injection, the device comprising:
a body comprising an open end, an interior wall extending inwardly from the open end, the interior wall partially defining a reservoir that extends into the body;
a cover removably sealing and closing the open end of the body, the cover and interior wall enclosing the reservoir;
a cooling medium in the reservoir; and
means for retaining the cooling medium in the body when the cover is removed;
the body, cover and cooling medium capable of being cooled to an application temperature substantially below room temperature;
whereby removing the cover exposes the cooled cooling medium such that placing the open end of the body against the skin causes sufficient heat transfer between the cooling medium and the skin to materially de-sensitize the skin.

2. The device of claim 1 wherein the cooling medium is solid at the application temperature.

3. The device of claim 1 wherein the cooling medium is sterile and the cover maintains a sterile environment within the reservoir prior to use.

4. The device of claim 1 wherein the cooling medium is liquid at room temperature.

5. The device of claim 4 wherein the cooling medium comprises water or a water mixture.

6. The device of claim 1 wherein the cover is formed from substantially planar sheet material.

7. The device of claim 1 wherein the body is a single piece of plastic.

8. The device of claim 1 wherein the open end of the body has an elongated shape or is “C”-shaped.

9. The device of claim 1 wherein the body comprises an applicator portion at the open end of the body to be pressed against the skin and a handle portion defining a handle at the other end of the body, a substantial portion of the handle away from the cooling medium to enable grasping the handle without substantial heat transfer through the handle to the cooling medium.

10. The device of claim 1 wherein the open end of the body is sized such that the device is capable of simultaneously cooling a plurality of injection sites on the skin.

11. The device of claim 1 wherein the cover is a first cover and the cooling medium incorporates a second cover closing the open end of the body, the second cover sealingly mounted on the body and remaining on the body after the first cover is removed.

12. The device of claim 1 wherein the retaining means comprises structure extending inwardly into the reservoir from the body wall and engaging the cooling medium.

13. The device of claim 1 wherein the open end of the body is sized such that the device is capable of cooling an area of skin away from the an injection site and infiltrated by medicant.

14. A method of reducing pain generated by hypodermic injections at a plurality of injection sites in human skin, the method comprising the steps of:
(a) applying a cooling medium against an area of skin, the perimeter of the area enclosing the plurality of injection sites, the cooling medium capable of cooling the skin sufficiently to simultaneously de-sensitize each of the injection sites;
(b) maintaining the cooling medium against the area of skin for a length of time sufficient to materially de-sensitize the entire area of skin; and
(c) serially administering a hypodermic injection at each injection site while the area of skin remains de-sensitized without additional application of a cooling medium between injections.

15. The method of claim 14 wherein step (a) comprises the step of applying ice in contact with the skin.

16. The method of claim 15 wherein the applied ice is sterile ice.
